(19) United States
(12) Patent Application Publication
(10) Pub. No.: US 2021/0275837 A1
(43) Pub. Date: Sep. 9, 2021

(54) BREATHABLE MASK WITH INTEGRATED GOGGLES AND AN AIR FILTER

(71) Applicants: Xing Li, Coquitlam (CA); Haoyu Li, New York, NY (US)

(72) Inventors: Xing Li, Coquitlam (CA); Haoyu Li, New York, NY (US)

(21) Appl. No.: 17/190,563

(22) Filed: Mar. 3, 2021

Related U.S. Application Data
(60) Provisional application No. 62/984,494, filed on Mar. 3, 2020.

(51) Int. Cl. A62B 7/10; A61F 9/02; A62B 23/06; A62B 18/04; A62B 18/08; A62B 23/02

(52) U.S. Cl. CPC A62B 7/10; A61F 9/027; A62B 23/06; A62B 7/02; A62B 18/084; A62B 18/086; A62B 23/02; A62B 18/04

(57) ABSTRACT

A breathing mask is provided. The breathing mask is secured around the head of the wearer and includes a nose lifter disposed on the front face of the breathing mask. The breathing mask includes a hood, a pair of eye goggles, a mouth aperture, and a check valve disposed on the rear lace of the hood. The eye goggles are affixed to the hood with an elastic band attached in the rear of the hood. Moreover, the mouth aperture is disposed on the front face of the hood below the nose filter. The hood is secured to the head of the wearer by a zipper disposed on the rear face of the hood and an elastic band encircling a pair of side faces and a rear face of the hood. The elastic band encircling the hood provides an airtight seal to protect the wearer from airborne diseases and other hazards.

### CROSS REFERENCE TO RELATED APPLICATIONS

This application claims the benefit of U.S. Provisional Application No. 62/984,494 filed on Mar. 3, 2020. The above identified patent application is herein incorporated by reference in its entirety to provide continuity of disclosure.

### BACKGROUND OF THE INVENTION

The present invention relates to breathing masks. More specifically, the present invention provides a breathing mask that protects the whole head of the wearer from any airborne diseases or similar hazards and includes a nose filter to filtrate the breathing air for the wearer.

When there is potential for human exposure to airborne diseases and other similar hazards, one way to reduce their risk of harm is to wear a facial mask. Such traditional masks filter the air breathed by the mask wearer to diminish their chances of contracting any airborne disease or suffering any complications from similar hazards. While these masks can provide the wearer some protection against the disease or hazard, some of them cannot be worn multiple times.

Moreover, since bacteria and viruses likely are to be absorbed in the air filtration system of the mask, during even a single use traditional masks can become contaminated. Attempting to clean or sterilize a mask can damage the air filtration system, rendering the mask defective. When the mask is defective, the wearer will not have the proper protection against the airborne diseases and similar hazards. Thus, wearing a mask with a defective air filtration system will provide little to no protection for the wearer against any airborne disease or similar hazards.

Following contamination, some of the traditional masks allow the air filtration system to be replaced. However, the traditional masks are lacking a proper method to clean or sterilize the entire mask. Without a method to clean or sterilize the entire mask, a mask wearer must dispose of the contaminated mask and find a new, uncontaminated mask. The new, uncontaminated mask will lower the risk of contracting an airborne disease or limit the exposure to a similar hazard when the wearer has any subsequent need to be in a potentially harmful environment. The constant need to replace the entire mask when they become contaminated can become wasteful.

Additionally, airborne diseases and other similar hazards can result in harm beyond just entering a person's respiratory system. Exposure of a disease or hazard to the eyes ears, or skin can also endanger the health of a person. However, traditional masks only protect the wearer from any exposure that enters their body by way of their mouth and nose. This leaves the rest of the head exposed to the hazards when traditional masks are in use. To supplement the traditional masks, wearers may attempto wear separate eye protection and separate hoods to limit a diseases or hazard exposure to the other parts of their head. When wearers piecemeal together traditional masks and other types of protection, there are high risks of leakage around the joints and connections between such components.

Therefore, there is a defined need amongst the known prior art references for a breathing mask that provides enhanced protection for the entire head of the wearer while being reusable.

### SUMMARY OF THE INVENTION

In view of the foregoing disadvantages inherent in the known types of breathing mask now present in the known art, the present invention provides a new breathing mask wherein the hood can be properly sterilized and cleaned and that the nose filter can be replaced when the current one can no longer effective filter the air to prevent airborne diseases and similar hazards from entering the respiratory system of the wearer.

It is therefore an object of the present invention to provide a breathing mask that includes a hood comprising airtight materials that covers the whole head of a person above the neck. The hood can be cleaned and sterilized for the wearer to use repetitively. Additionally, the hood is held together by a closure fastener disposed on the rear face of the hood. The closure fastener can be selectively engaged and disengaged. Moreover, the closure fastener extends along the rear face is located at the bottom of the rear face of the hood. Furthermore, the hood is secured to the head of the wearer by an elastic band encircling a pair of side faces and a rear face of the hood.

Another aspect of the present invention is related to a nose filter. The nose filter includes a ring and a plurality of layers of filtration material to prevent any airborne toxins from reaching the nose of the wearer. The ring comprises a threaded interface that includes a first threading deposed on the nose filter and a second threading disposed on the front fact of the hood. To secure the nose filter to the hood, the first threading and the second threading are coupled together. Moreover, when the nose filter has been used, the wearer can separate the nose filter and replace it with a new nose filter. In some embodiments, the second threading can also connect to a portable oxygen bottle or other respiratory assistant devices.

Yet another aspect of the present invention is a mouth aperture disposed on the front face of the hood below the nose filter. The mouth aperture comprises a closure fastener. The closure fastener can be selectively engaged and disengaged by the wearer. The wearer can selectively disengage the closure fastener if they desire access to their mouth for eating or drinking while they are wearing the breathing mask.

Still another aspect of the present invention is a pair of eye goggles disposed on the front face of the hood above the nose filter. The opposing ends of the pair of eye goggles are affixed by an elastic band. The elastic band further secures the breathing mask to the head of the wearer. The elastic bands that are affixed to opposing ends of the pair of goggles secure to the hood by channels that are on opposing sides of the hood. The elastic band enters through the inlet of the channel located on the front face of the hood and exits through the outlet of the channel located on the rear face of the hood. After exiting the channel, the elastic bands are affixed to one another using a securing fastener. One embodiment of the securing fastener is a buckling system.

In some embodiments, the present invention has a visual recording device disposed above the pair of eye goggles and an auditory component that is disposed on the side of the hood. The auditory component is proximal to the channel disposed on the side of the hood. The visual recording device and the auditory component provide the wearer with an ability to store recordings and amplify sounds while wearing the breathing mask.

Another aspect of the present invention is a check valve disposed on the rear face of the hood. The check valve removes the buildup of moisture and releases pressure that accumulates underneath the hood while wearer is utilizing the breathing mask.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a front view of an embodiment of the breathing mask.

FIG. 2 shows a rear view of an embodiment of the breathing mask.

FIG. 3 shows a perspective view of the replaceable nose filter component of an embodiment of the breathing mask.

FIG. 4 shows a side view of an embodiment of the breathing mask.

FIG. 5a shows a side view of an embodiment of the breathing mask with an oxygen supply attachment.

FIG. 5b shows a side view of an embodiment of the breathing mask with an alternate attachment.

### DETAILED DESCRIPTION OF THE INVENTION

Reference is made herein to the attached drawings. Like reference numerals are used throughout the drawings to depict like or similar elements of the breathing mask. For the purposes of presenting a brief and clear description of the present invention, the preferred embodiment will be discussed as being used to prevent airborne disease and similar hazards from infiltrating the respiratory system of the wearer. The figures are intended for representative purposes only and should not be limiting in any respect.

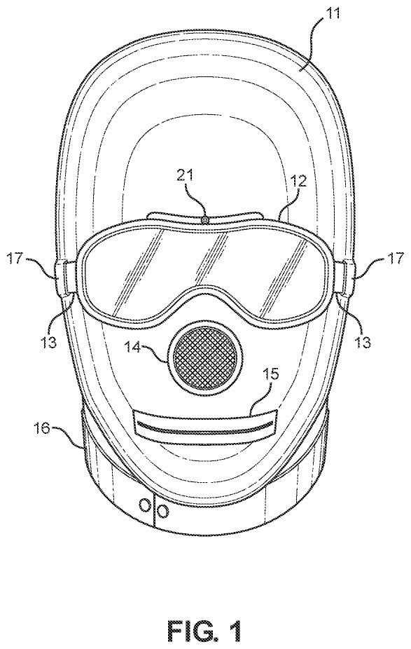

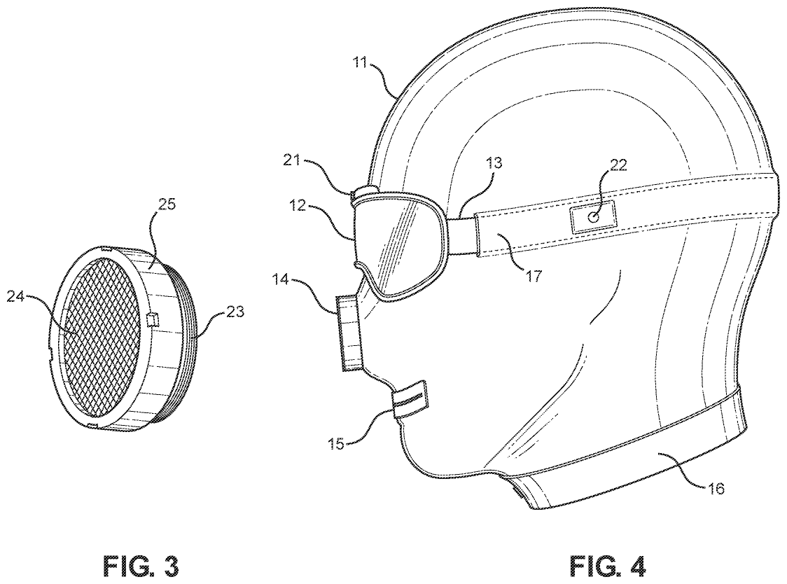

Referring now to FIG. 1, there is shown a front view of an embodiment of the breathing mask. The breathing mask comprises a hood 11 and a replaceable nose filter 14. The hood 11 is configured to extend to cover the whole head of a wearer. The hood 11 is airtight to limit the wearer from exposure to any airborne toxins or diseases. The replaceable nose filter 14 is configured to filter the air that the wearer breathes. The replaceable nose filter 14 is disposed on the front face of the hood 11. In some embodiments the nose filter 14 is a round, replaceable system composed of a plurality of layers of filter material (visible in FIG. 3). A mouth aperture 15 is disposed on the front side of the hood 11 below the nose filter 14. The mouth aperture 15 comprises a closure fastener. The closure fastener can be selectively engaged and disengaged by the wearer. In one embodiment of the breathing mask, the closure fastener is a zipper. In another embodiment of the breathing mask, the closure fastener is a hook and loop fastener. At the bottom of the hood 11, a neck band 16 comprises a plurality of layers of elastic bands. The neck band 16 enables the wearer to maintain an airtight seal around their neck and secures the hood 11 to the head. Moreover, a pair of eye goggles 12 are disposed on the front face of the hood above the nose filter 14. An eye goggle band 13 affixed to a pair of opposing ends of the pair of eye goggles 12. The eye goggles 12 provide protection of the eyes of the wearer. Additionally, in some embodiments, the breathing mask includes a camera or other visual recording device 21 disposed above the pair of eye goggles 12. The visual recording device 21 captures and records images from the perspective of the wearer and saves the data to a corresponding memory bank within the breathing mask, which can be accessed via an external computer.

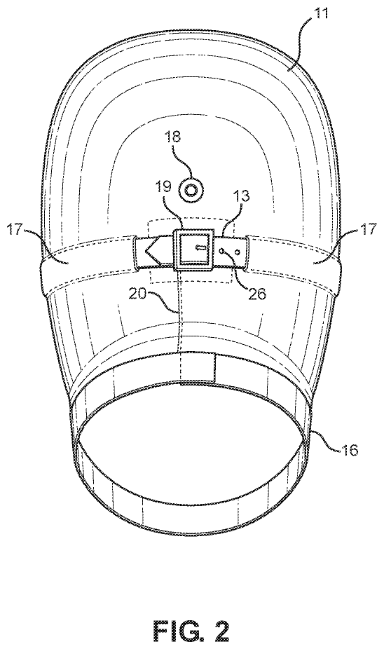

Referring now to FIG. 2, there is shown a rear view of an embodiment of the breathing mask. The breathing mask comprises a hood 11 that can be secured around the head of a wearer. The eye goggle bands 13 that connect to opposing ends of the eye goggles 12 are affixed to the hood 11, such that the eye googles 12 cannot be easily separated from the hood 11. There is a plurality of channels 17 disposed on opposing sides of the hood 11 and the eye goggle bands 13 are inserted into the channels 17. The eye goggle bands 13 engage the channels 17 at the inlet by the eye googles 12 and disengage of the channels 17 at the outlet on the rear of the hood 11 where the opposing ends of the eye goggle bands 13 are joined by a securing fastener. One embodiment of the securing fastener for the eye goggle bands 13 that are disposed to the eye goggles 12 comprises a buckle 19 and a plurality of buckle holes 26. In this embodiment, the buckle 19 and the plurality of buckle holes 26 are on opposing eye goggle bands 13. Moreover, at least one buckle hole 26 is in position to engage with the buckle 19 to secure the opposing eye goggle band 13 disposed on the eye goggles 12. Additionally, a check valve 18 is disposed on the rear of the hood 11 above the securing fastener for the eye goggle bands 13. An adhesive zipper 20 is disposed on the rear side of the hood 11. The adhesive zipper 20 secures the hood 11 about the head. The adhesive zipper 20 is disposed on the lower part of the rear hood from the neck band 13 to the eye goggle bands 13 securing fastener. The adhesive zipper 20 extends along the rear face of the hood 11 configured so the zipper can be selectively engaged and disengaged. Moreover, the plurality of zipper is positioned at the bottom of the rear face of the hood 11.

Referring now to FIG. 3, there is shown a perspective view of the replaceable nose filter 14 when it is not disposed in the proper position on the front face of the hood 11. The nose filter 14 comprises a plurality of layers of filtration material 24 and a ring 25. The ring 25 provides a supporting frame for the plurality of layers of filtration materials 24. The plurality of layers of filtration material are disposed on top of one another. Additionally, the plurality of layers of filtration material 24 is design to absorb the airborne toxins and diseases. Moreover, the plurality of layers of filtration material 24 prevents airborne toxins and diseases from entering the respiratory system of the wearer. The plurality of layers of filtration material 24 is composed of the same material as a medical mask. One example of the plurality of layers of filtration material 24 is carbon. Another example of the plurality of layers of filtration material 24 is polypropylene. The ring 25 comprises a threaded interface, a first threading deposed on the nose filter 23, and a second threading disposed on the front face of the hood 11. The threaded interfaces connect by screwing the first threading on the nose filter 23 into the second treading on the front face of the hood 11.

Referring now to FIG. 4, there is shown a side view of an embodiment of the breathing mask. In some embodiments of the breathing mask, there is an auditory component 22 that is disposed on the side of the hood 11 proximal to the channel 17. The auditory component 22 provides an amplification of the sound that the auditory component 22 receives and permits the wearer to pair the auditory component 22 to an electronic device.

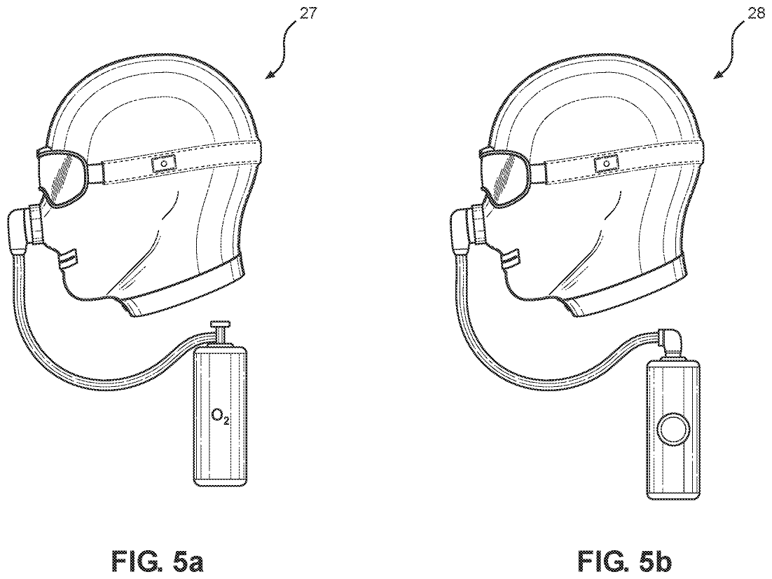

Referring now to FIGS. 5a and 5b, there are shown embodiments of the breathing mask with different attachments. One embodiment shown includes the attachment of a respirator aid 28. The respirator aid 28 is comprises a tubing with an attaching end that has a threaded interface. The respirator aid 28 is joined with the hood 11 by engaging the threaded interface on the attaching end with the second threading. Another embodiment includes the attachment of an oxygen source 27. The oxygen source 27 is comprises a tubing with an attaching end that has a threaded interface. The oxygen source 27 is joined with the hood 11 by engaging the threaded interface on the attaching end with the second threading.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to falling within the scope of the invention

We claim:

1. A breathing mask, comprising:
a hood comprising a front face, a pair of side faces, and a rear face defining a cavity dimensioned to receive a head of a wearer;
a nose filter centrally disposed on the front face of the hood;
a mouth aperture disposed on the front face of the hood below the nose filter;
a pair of eye goggles disposed on the front face of the hood above the nose filter;
an elastic band encircling the pair of side faces and the rear face of the hood;
and elastic band affixed to a pair of opposing ends of the pair of eye goggles;
a zipper disposed on the rear face of the hood;
a plurality of channels disposed on the hood, wherein the plurality of channels comprise a pair of eye goggle channels on the front face of the hood and one or more outlet channels on the rear face of the hood; and
a check valve disposed on the rear face of the hood.

2. The breathing mask of claim 1, wherein the nose filter further comprises a ring and a plurality of layers of filter material.

3. The nose filter of claim 2, wherein the ring further comprises a first threading interface.

4. The breathing mask of claim 1, wherein a second threading interface is disposed on the front face of the hood.

5. The breathing mask of claim 1, wherein the nose lifter connects to the hood by engaging the first threading interface of the nose filter with the second threading interface on the front face of the hood.

6. The breathing mask of claim 1, wherein the mouth aperture comprises a zipper system.

7. The breathing mask of claim 6, wherein the mouth aperture zipper system comprises a closure fastener extending along the mouth aperture, wherein the closure fastener can be selectively engaged and disengaged.

8. The breathing mask of claim 1, wherein one or more elastic bands encircling a pair of side faces and a rear face of the hood is disposed towards the bottom of the hood, closer to the upper neck of the wearer.

9. The breathing mask of claim 1, wherein the elastic bands affixed to a pair of opposing ends of the pair of eye goggles enter the channel by the inlet and exit the channel by the outlet.

10. The breathing mask of claim 1, wherein the one elastic band affixed to opposing ends of the pair of eye goggles further comprise a securing fastener disposed on a first end of the elastic band and a plurality of holes disposed on a second end of the elastic band.

11. The one elastic band of claim 1, wherein the securing fastener engages one of the plurality of holes to secure the opposing ends of the elastic band.

12. The breathing mask of claim 1, wherein the zipper disposed on the rear face of the hood extends along rear face of the hood below the securing fastener.

13. The breathing mask of claim 1, wherein the zipper disposed on the rear face of the hood extends along rear face of the hood can be selectively engaged and disengaged.

14. The breathing mask of claim 1, wherein some embodiments have a visual recording device disposed above the pair of eye goggles and an auditory component that is disposed on the side of the hood proximal to the channel, the auditory component configured to amplify incoming sounds for the wearer.
